(19) United States
(12) Patent Application Publication
(10) Pub. No.: US 2024/0231490 A1
(43) Pub. Date: Jul. 11, 2024

(54) BRAIN-COMPUTER INTERFACE FOR CONTROLLING MOVEMENT OF AN EFFECTOR

(71) Applicant: Commissariat à l'Energie Atomique et aux Energies Alternatives, Paris (FR)

(72) Inventors: Felix MARTEL, Grenoble Cedex 09 (FR); Hafid SID-AHMED, Grenoble Cedex 09 (FR); Tetiana AKSENOVA, Grenoble Cedex 09 (FR)

(73) Assignee: Commissariat à l'Energie Atomique et aux Energies Alternatives, Paris (FR)

(21) Appl. No.: 18/398,343

(22) Filed: Dec. 28, 2023

(30) Foreign Application Priority Data
Dec. 29, 2022 (FR) 22 14664

(51) Int. Cl. G06F 3/01; B25J 9/16

(52) U.S. Cl. CPC G06F 3/015; B25J 9/161

(57) ABSTRACT

The invention relates to a method for controlling the movement, through a space, of an effector via a brain-computer interface, comprising:
a) acquiring electrophysiological signals produced in the cortex of an individual, at a measurement time;
b) processing the electrophysiological signals, to form input data;
c) processing the input data, by means of a predictive model implemented by the brain-computer interface, to define an effector movement (xₚ^{s}) at the measurement time;
d) determining a corrected effector movement (x_{c}^{s+1}−x_{c}^{s}), based on the movement (xₚ^{s}) defined by the predictive model in step c);
e) moving the effector through the space, by means of the brain-computer interface, based on the corrected movement resulting from step d);
f) reiterating steps (a) to (e), the method comprising, in each step d), estimating the target position [symbol] , at the measurement time. FIG. 2.

### TECHNICAL FIELD

The invention relates to a brain-computer interface (BCI) intended to allow an automated effector (for example a robot or an exoskeleton) to be controlled via cortical electrophysiological signals.

### PRIOR ART

The field of brain-computer interfaces is undergoing rapid development and appears to be an attractive way of allowing people with disabilities to control effectors with their thoughts. It is a question of recording, generally using cortical electrodes, electrophysiological signals. The latter are processed by algorithms allowing a control signal to extracted, with a view to controlling effectors. The control signal may allow an effector—such as an exoskeleton, orthotic device, computer or robot—to be controlled with a view to providing assistance to the user. The algorithms employed allow an instruction given by the user to be interpreted, this instruction being captured, by the electrodes, in the form of signals that are said to electrophysiological because they are representative of the electrical activity of neurons. This electrical activity is generally measured in the cortex, by means of cortical electrodes placed in the skull. It may also be measured by electroencephalographic electrodes, which are less intrusive because they are arranged on the scalp, but also less effective, especially from the point of view of spatial resolution. Another solution is to record electrophysiological signals via magnetoencephalography, this requiring a dedicated apparatus.

The algorithms employed are generally based on a predictive model. The predictive model uses input data obtained by pre-processing the recorded electrophysiological signals. An example of calibration of a predictive model is described in U.S. Pat. No. 11/497,429. The predictive model allows input data to be processed to establish a predicted movement, allowing an effector movement corresponding to the detected electrophysiological signals to be made.

However, use of a predictive model may be accompanied by a certain level of imprecision, when the user seeks to move the effector towards a predetermined target position. The invention allows this problem to be solved.

### SUMMARY OF THE INVENTION

A first subject of the invention is a method for controlling the movement, through a space, of an effector via a brain-computer interface, the movement tending to bring the effector closer to a target position, comprising the following steps:
a) acquiring electrophysiological signals produced in the cortex of an individual, at a measurement time, the effector occupying a position, in the space, at said measurement time;
b) processing the electrophysiological signals, to form input data;
c) processing the input data, by means of a predictive model implemented by the brain-computer interface, to define an effector movement at the measurement time;
d) determining a corrected effector movement, based on the movement defined by the predictive model in step c);
e) controlling the movement of the effector through the space, by means of the brain-computer interface, based on the corrected movement resulting from step d);
f) incrementing the measurement time and reiterating steps a) to e) until a criterion for ending the iterations is met;
the method comprising, in each step d) following at least a first iteration of steps a) to e):
di) estimating the target position, at the measurement time, based on at least:
   an effector position at the measurement time and at least one time preceding the measurement time;
   a movement defined by the predictive model at the measurement time and at least at the time preceding the measurement time;
dii) based on the target position estimated in sub-step di), determining a movement towards the estimated target;
diii) taking into account the movement towards the estimated target, resulting from sub-step dii), to establish the corrected movement at the measurement time.
Sub-step di) may comprise:
computing a target probability density, corresponding to a probability density reflecting the probability that each point in the space corresponds to the target position;
determining the point in the space maximizing the target probability density, or a quantity comprising the target probability density, the point thus determined corresponding to the estimated target position.

Computation of the target probability density may take into account:
a plurality of effector positions at times preceding the measurement time;
a plurality of movements defined by the predictive model at times preceding the measurement time.

The target probability density may be computed based on a combination, e.g. a linear combination,
of a first conditional probability density dependent on:
   effector positions at times preceding the measurement time;
   movements defined by the predictive model at times preceding the measurement time;
and of a second conditional probability density dependent on:
   the effector position at the measurement time;
   the movement defined by the predictive model at the measurement time.

The first conditional probability density may be weighted by a forgetting factor.

Sub-step di) may comprise determining the point maximizing a quantity comprising the target probability density, the quantity corresponding to the target probability density decreased:
by a first distance, weighted by a first regularization factor, between each point and the target position estimated at a preceding measurement time;
and/or by a second distance, weighted by a second regularization factor between each point and the effector position at the measurement time.

In sub-step dii), the movement towards the estimated target may be established so that the interface moves the effector from the position at the measurement time to the target position estimated at the measurement time.

In sub-step diii), the corrected movement may be a combination, for example a linear combination, of the movement defined by the predictive model in step b) and of the movement towards the estimated target resulting from sub-step dii). This linear combination may employ:
a first weighting factor, applied to the movement defined by the predictive model;
a second weighting factor applied to the movement towards the estimated target.

A second subject of the invention is a brain-computer interface, comprising:
sensors, able to detect electrophysiological signals representative of a cortical activity;
an effector, configured to be actuated by a control signal generated by the brain-computer interface;
a processor programmed to determine an effector movement depending on input data resulting from the detected electrophysiological signals;
   the processor being configured to implement steps c) to f) of a method according to the first subject of the invention, at various measurement times.

The invention will be better understood on reading the description of the examples of embodiment that are presented, in the rest of the description, with reference to the figures listed below.

### FIGURES

FIG. 1 schematically shows the main components of a brain-computer interface.

FIG. 2 shows an effector movement in a 3D environment.

FIG. 3 illustrates a vector corresponding to a movement predicted by a predictive model; a desired movement, between the effector position and the target position; and the angular error between the two vectors.

FIG. 4 shows the main steps of a method implemented by a brain-computer interface according to the invention.

FIGS. 5A and 5B show the variation in distances between the effector and the target, and between the estimated target and the actual target, as a function of time, considering two different noise models. In FIGS. 5A and 5B, the effector is moved towards the target without taking into account the target positions estimated over time.

FIGS. 6A and 6B show the variation in angular accuracies as a function of time, considering two different noise models.

FIGS. 7A to 7D show the influence of weighting the effector movement between the movement predicted by the interface and a corrected movement, with implementation of the invention, as a function of an estimation of the target position.

FIG. 8 illustrates an implementation of the invention in 3D.

### DESCRIPTION OF PARTICULAR EMBODIMENTS

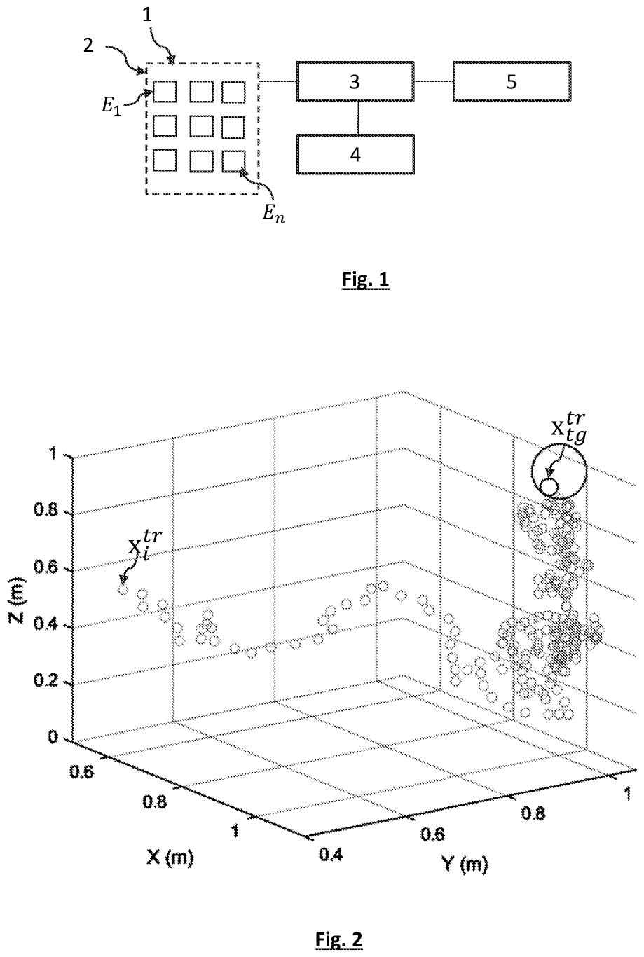

FIG. 1 shows the main elements of a brain-computer interface 1 according to the invention. It is a question of a device comprising sensors 2, allowing electrophysiological signals representative of neuronal activity to be acquired. The sensors 2 are, for example, cortical electrodes E₁ . . . Eₙ. The sensors 2 are connected to a processor 3 by a wired or wireless link. The processor 3 is able to execute instructions coded in a memory 4. These instructions in particular comprise algorithms allowing a control signal intended to drive an effector 5 to be generated.

The effector 5 is an actuator able to perform an action under the effect of the control signal addressed to it. It may be an exoskeleton, a robot, or a computer. The effector is then controlled by the electrophysiological signals acquired by the cortical electrodes 2, via an algorithm coded in the memory 4 and implemented by the processor 3. More precisely, the objective of the device is to allow the effector to be moved, through an environment, to a target position.

The environment may be a real environment, the effector for example being a robotic arm, a robotic orthotic device, a glove or a tool carried by the user's hand. The objective is to move the effector to the target position, which is a position that the user wishes to reach. The environment may be virtual, the effector for example being a computer mouse. The user may then drive a cursor to a determined position on a screen, so as to activate a command. The target position is the position of the cursor that the user wishes to reach.

The device 1 is a brain-computer interface, in the sense that it allows the effector 5 to be controlled by the one or more electrophysiological signals measured by the sensors 2. The electrophysiological signals may be acquired by cortical electrodes placed in contact with the sensorimotor cortex, magnetoencephalography sensors or electroencephalography electrodes.

The control signal is formed by the processor 3 based on the electrophysiological signals. The algorithm implements a predictive model, which generates an output signal, which allows the effector 5 to be controlled. The predictive model may be determined as described in patent U.S. Pat. No. 11,497,429. More precisely, the output signal allows the effector 5 to be moved to the target position according to a movement predicted by the predictive model.

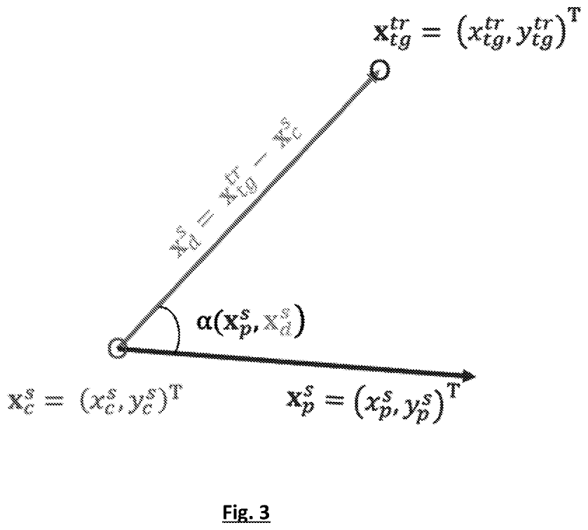

The space may be discretized into a plurality of points. FIG. 2 illustrates a gradual movement of an effector between an initial position xᵢ^{tr} and a target position x_{ig}^{tr}. The exponent tr designates the attempt in question. The movement is performed in steps the size of which depends on the sampling frequency of the interface, which may be 10 Hz. The sampling frequency is preferably high enough to prevent the movement from appearing too jerky to the user, and to limit the reaction time of the system when the user changes her or his intention. At each measurement time s, the effector occupies a discrete position x_{c}^{s}, x_{c}^{s} is a point represented by a vector the dimension of which corresponds to the dimension of the space through which the effector is moved. In a first embodiment, a 2-dimensional space is considered. Each vector is defined by a coordinate x and a coordinate y.

At each time s, the predictive model of the interface generates a predicted movement xₚ^{s}, which corresponds to the movement corresponding to the electrophysiological signals detected by the interface. xₚ^{s}=x_{c}^{s+1}−x_{c}^{s} (1). The movement xₚ^{s}corresponds to the effector movement, as predicted by the predictive model, between two successive times s, s+1, depending on the electrophysiological signals detected at the time s.

At each time s, a vector xa that corresponds to a desired movement may be determined, such that:

$${x}_{d}^{s} = {x}_{t g}^{t r} - {x}_{c}^{s} \qquad ( 2 )$$

However, the position x_{tg}^{tr}, although known by the user, is not known by the interface. It is therefore necessary to estimate it, as described below, so as to be able to determine x_{d}^{s}. x_{d}^{s} corresponds to an ideal movement vector, i.e. the one that would be followed by the effector if the interface were perfect. It is assumed that the user intends to move the effector towards the target in a straight line.

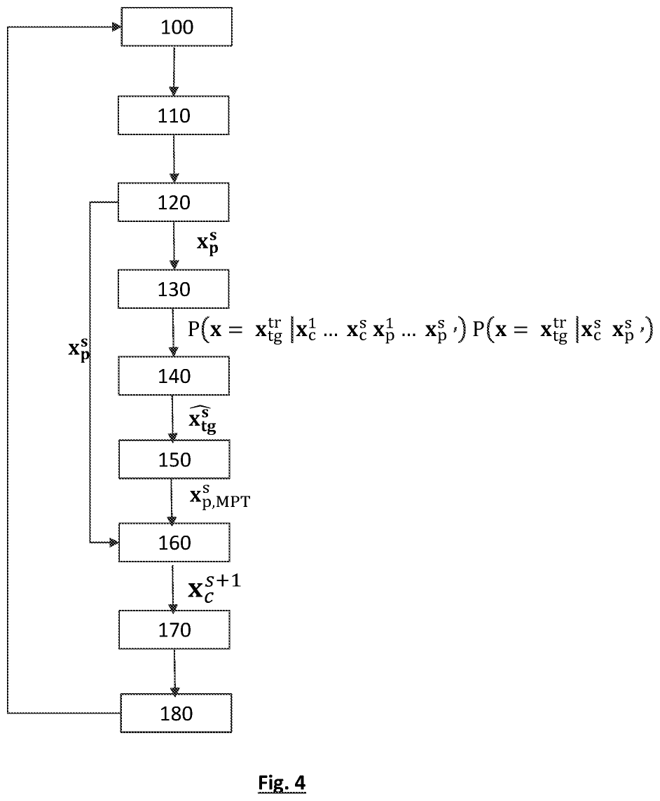

FIG. 3 shows the vectors x_{d}^{s} and xₚ^{s}. The angle between the two vectors x_{d}^{s} and xₚ^{s} corresponds to an angular error, denoted α(xₚ^{s}, xₚ^{d}).

An important element of the invention is estimation, by the interface, at each measurement time s, of the target position [symbol] . The estimation of the target position is obtained by maximizing a probability, called the target probability, defined at every point in the space, and corresponding to the probability that each point in the space corresponds to the target xₜₕ^{tr}.

The target probability is a conditional probability that is defined at a plurality of points in the space, and preferably at every point in the space. Assuming that the user wishes to move the effector in a straight line to the target position, and therefore is seeking to reduce angular error, it may be estimated that, at each point in the space, represented by a vector x, of coordinates (x, y). The target probability density is :

$$P ( x = {x}_{tg}^{tr} \vert {x}_{c}^{s} , {x}_{p}^{s} ) = \frac{1}{\sqrt{2 {πσ}^{2}}} {e}^{- \frac{{α ( {x}_{p}^{s} , x - {x}_{c}^{s} )}^{2}}{2 {σ}^{2}}} \qquad ( 3 )$$

The angular error α(xₚ^{s}, X−x_{c}^{s}) is a variable having a gaussian probability density, of variance σ².

The target probability density P(x=x_{tg}^{tr}|x_{c}^{s}, xₚ^{s}) is estimated at each measurement time s.

The variance σ² may be estimated as described below.

Advantageously, the target probability density may be estimated by taking into account the effector positions x_{c}^{s=1} . . . x_{c}^{s} and the movements predicted by the predictive model xₚ^{s=1} . . . xₚ^{s} at times preceding the measurement time. Thus, the target probability density is such that:

$$P ( x = {x}_{tg}^{tr} \vert {x}_{c}^{1} … {x}_{c}^{s} , {x}_{p}^{1} … {x}_{p}^{s} ) = \frac{1}{s} {\sum }_{i = 1}^{s} P ( x = {x}_{tg}^{tr} \vert {x}_{c}^{i} , {x}_{p}^{i} ) \qquad ( 4 )$$

For a point of position x, the probability density according to expression (4) corresponds to an average of the target probabilities established over time for said point.

At each time s, the target position is considered to be the position maximizing the target probability density.

$$= arg {max}_{x} [ P ( x = {x}_{tg}^{tr} \vert {x}_{c}^{1} … {x}_{c}^{s} , {x}_{p}^{1} … {x}_{p}^{s} ) ] \qquad ( 5 )$$

At each time s, the estimation of the target position [symbol] used to control the effector movement. A movement towards the estimated target, x_{p,MPT}^{s}, is established, such that:

$${x}_{p , MPT}^{s} = - {x}_{c}^{s} \qquad ( 6 )$$

x_{p,MPT}^{s} is the vector between the current position x_{c}^{s} and the estimated target position [symbol] . It is the vector of the movement towards the estimated target.

x_{d}^{s}, defined in (2), is the vector between the current position x_{c}^{s} and the actual target position x_{tg}^{s}. It is the desired movement vector.

The effector position at the time x_{c}^{s+1} may be estimated via the expression:

$${x}_{c}^{s + 1} = β ( k \frac{{x}_{p}^{s}}{‖ {x}_{p}^{s} ‖} + ( 1 - k ) \frac{{x}_{p , MPT}^{s}}{‖ {x}_{p , MPT}^{s} ‖} ) + {x}_{c}^{s} \qquad ( 7 )$$

where k is a first weighting factor and 1'k is a second weighting factor. In this example, 0≤k≤1. β is a positive real number, corresponding to a previously determined distance of movement. Setting k=1 amounts to not taking into account the estimated target position, this resulting in a movement according to the prior art. Setting k=0 amounts to taking into account only the target estimation in the control of the movement.

The first weighting factor k and the second weighting factor 1−k make it

possible to proportion the contributions:
of the movement predicted by the predictive model of the interface;
of the movement towards the estimated target position.

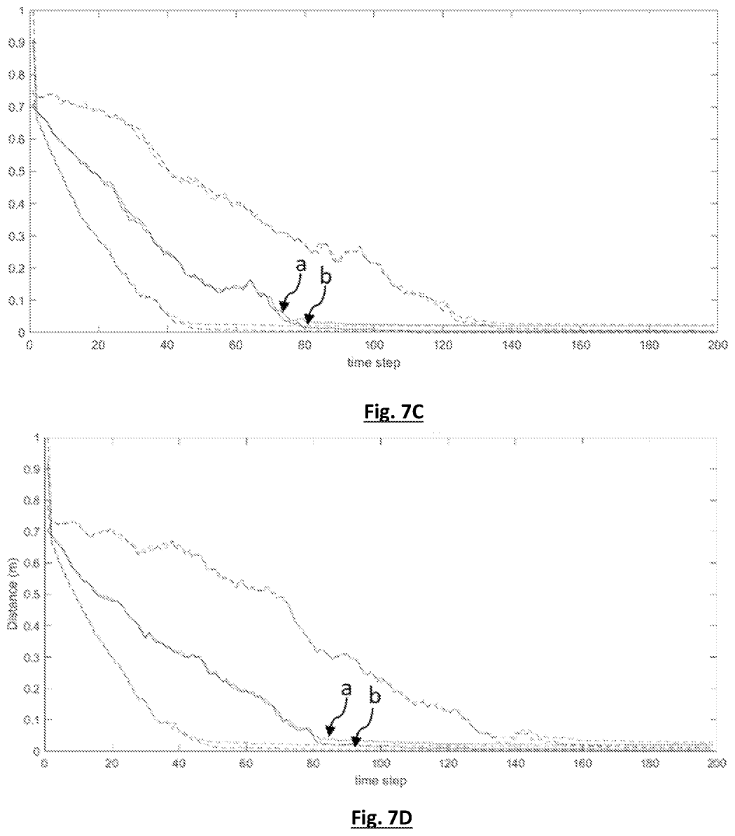

The influence of these weighting factors is discussed with reference to FIGS. 7A to 7D.

The movement x_{c}^{s+1}−x_{c}^{s} corresponds to a corrected movement, taking into account both the movement predicted by the predictive model, and the movement towards the estimated target position. This is a key aspect of the invention.

Thus, the effector movement is partly defined by the predictive model, as in the prior art, and partly defined depending on the estimated target position [symbol] . Taking the estimated target position into account, when determining the effector movement, is an important aspect of the invention.

According to one variant, the target probability density is such that:

$$P ( x = {x}_{tg}^{tr} \vert {x}_{c}^{1} … {x}_{c}^{s} , {x}_{p}^{1} … {x}_{p}^{s} ) = \frac{\begin{matrix}( s - 1 ) \cdot μ \cdot P ( x = {x}_{tg} \vert {x}_{c}^{1} … \\ {x}_{c}^{s - 1} , {x}_{p}^{1} … {x}_{p}^{s - 1} ) + P ( x = {x}_{tg} \vert {x}_{c}^{s} , {x}_{p}^{s} )\end{matrix}}{s} \qquad ( 8 )$$

where μ is a positive real number, corresponding to a forgetting factor. μ allows the times before the current time s that are taken into account to be proportioned. μ is generally comprised between 0 and 1. μ allows the impact of old times in decision making to be modulated.

According to this variant, the target probability density is established based on a linear combination:
of a first conditional probability density P(x=x_{tg}|x_{c}¹ . . . x_{c}^{s-1}, xₚ¹ . . . xₚ^{s-1}), weighted by the forgetting factor μ, and dependent on the effector positions at times preceding the measurement time, and on movements predicted by the predictive model at times preceding the measurement time.
of a second conditional probability density P(x=x_{tg}|x_{c}^{s}, xₚ^{s}), dependent on the effector position x_{c}^{s} at the measurement time and on the movement predicted by the predictive model xₚ^{s} at the measurement time.

According to another variant, the target position, at each measurement time, is estimated using the expression:

$$( 9 )$$

$$= arg {max}_{x} [ P ( x = {x}_{tg} \vert {x}_{c}^{1} … {x}_{c}^{s} , {x}_{p}^{1} … {x}_{p}^{s} ) - {λ}_{1} ‖ - x ‖ - {λ}_{2} ‖ {x}_{c}^{s} - x ‖ ]$$

where λ₁ and λ₂ are positive scalars, which correspond to regularization factors. Here, it is a question of maximizing a quantity established based on the target probability density, decreased:
by a first distance, between the target position [symbol] and each point x in the space, said first distance being weighted by a first regularization factor λ₁. This prevents two estimated successive positions of the target from being too far apart;
and/or by a second distance between the effector position x_{c}^{s} at the measurement time and each point x in the space, said second distance being weighted by a second regularization factor λ₂. This prevents the estimated target position from being too far away from the effector position at the measurement time. This makes it possible to contain the target position in a certain workspace, around the current position.

Expression (9) may be implemented without taking into account the first distance (λ₁=0) or without taking into account the second distance (λ₂=0).

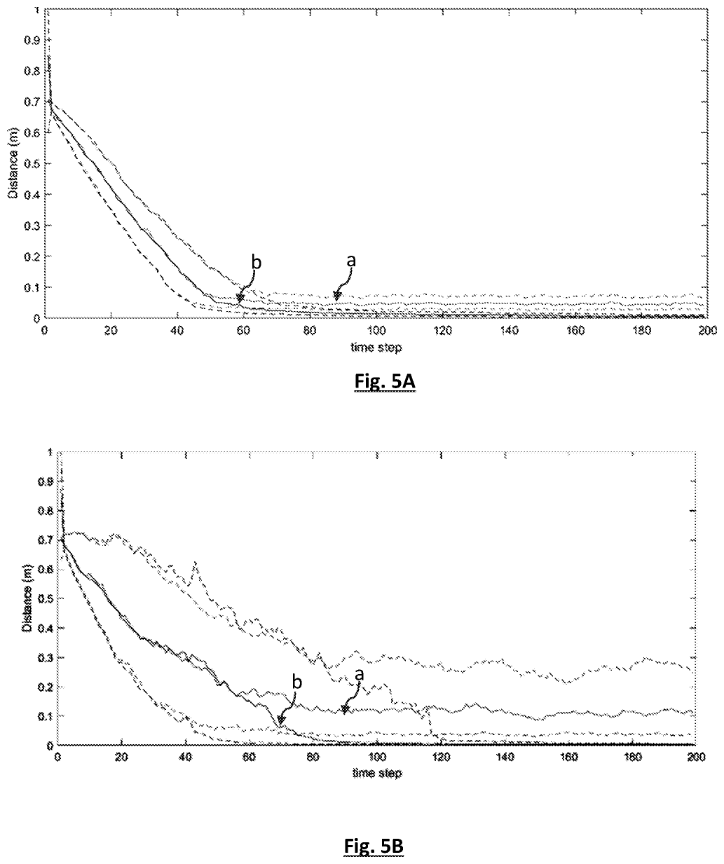

FIG. 4 schematically shows the main steps of a method implemented by a brain-computer interface according to the invention.

Step 100: taking into account electrophysiological signals, emitted by the user and detected by the sensors 2 at a time s.

Step 110: based on the detected electrophysiological signals, forming an input signal feeding the predictive model.

Step 120: defining, by means of the predictive model, the movement xₚ^{s} at the time s, depending on the input signal.

Step 130: determining a target probability density, P(x=x_{tg}^{tr}|x_{c}^{s} . . . x_{c}^{s}, xₚ¹ . . . xₚ^{s}), P(x=x_{tg}^{tr}|x_{c}^{s}, xₚ^{s}) at every point x in the space through which the effector is moved, depending on the effector position at the time s, on the predicted movement at the time s, and possibly depending on the effector position and on the predicted movement at times preceding the time s: cf. expressions (3), (4) or (8).

Step 140: estimating a position [symbol] maximizing the probability density resulting from step 130, or a quantity using the probability density resulting from step 130. Cf. expressions (5), (6) or (9).

Step 150: based on [symbol] , computing a movement towards the estimated target position xp,MPT

Step 160: based on the movement xₚ^{s} defined by the predictive model, which results from step 120, and on the movement towards the estimated target position, which results from step 150, determining a corrected movement x_{c}^{s+1}−x_{c}^{s}: cf. expression (7).

Step 170: moving the effector depending on the corrected movement.

Step 180: incrementing the time index s and reiterating steps 100 to 170 until the target is reached or considered to have been reached, or until the algorithm is intentionally stopped by the user.

### Determining σ

The standard deviation o is determined in advance in preliminary trials. During these trials, the position x_{tg}^{tr} of the target is known. It is possible to establish a cost function allowing an angular accuracy to be estimated, and to determine the value of σ that optimizes the cost function. The cost function may have an average cosine similarity function MCS such that:

$$MCS = \frac{1}{{N}_{ss}} {\sum }_{tr = 1}^{{N}_{ss}} \frac{1}{{s}_{tr}} {\sum }_{s = 1}^{{s}_{tr}} Cos Sim ( , {x}_{d}^{s} ) \qquad ( 11 )$$

where
Nₛₛ corresponds to a number of prior trials carried out;
sₜᵣ corresponds to the number of time increments during each trial tr;
CosSim(xₚ^{s}, x_{d}^{s}) corresponds to the cosine similarity function, such that:

$$Cos Sim ( {x}_{p}^{s} , {x}_{d}^{s} ) = \frac{, {x}_{d}^{s} \cdot }{‖ ‖ ‖ {x}_{d}^{s} ‖} ; = - {x}_{c}^{s} ; \qquad ( 11 ’ )$$

x_{d}^{s}=x_{tg}^{tr}−x_{c}^{s}. x_{d}^{s} corresponds to the desired movement.

The value of σ is the one that maximizes the cost function MCS.

The cost function may be an average error in the estimated target position with respect to the actual target position, over the last however many time steps, for example the last 30 time increments.

$$MDE = \frac{1}{{N}_{ss}} {\sum }_{tr = 1}^{{N}_{ss}} \frac{1}{30} {\sum }_{s = {s}_{tr} - 29}^{{s}_{tr}} ‖ - {x}_{tg}^{tr} ‖ \qquad ( 12 )$$

Nss and str were defined in connection with (11).

The value of o is the one that minimizes the cost function MDE.

### Simulations

Simulations were carried out on artificial data sets, in which the target position was known. Thus, at each time, x_{d}^{s} was known. Based on x_{d}^{s}, the movement predicted by the predictive model of an interface was predicted considering the movement predicted by the predictive model xₚ^{s} to be such that:

$${x}_{p}^{s} = {x}_{d}^{s} + {ε}^{s} \qquad ( 13 )$$

where ε^{s} is a vector representing noise.

Two different noise models were taken into account: the first noise model was

independent and identically distributed (IID) Gaussian noise of power η²=1.4. The second noise model was Gaussian noise dependent on the preceding time increments, such that:

$${ε}^{s} = {\sum }_{i = 1}^{10} 0.9 {5}^{10 - i} * {w}^{s - i} \qquad ( 14 )$$

where w follows a centred IID distribution of power η²=0.6.

The target probability density was computed considering σ²=3 rad².

A first series of trials was carried out k considering=1 in (7). During these trials, a target position was estimated in each time increment. The estimated target position was not used when controlling the movement of the effector.

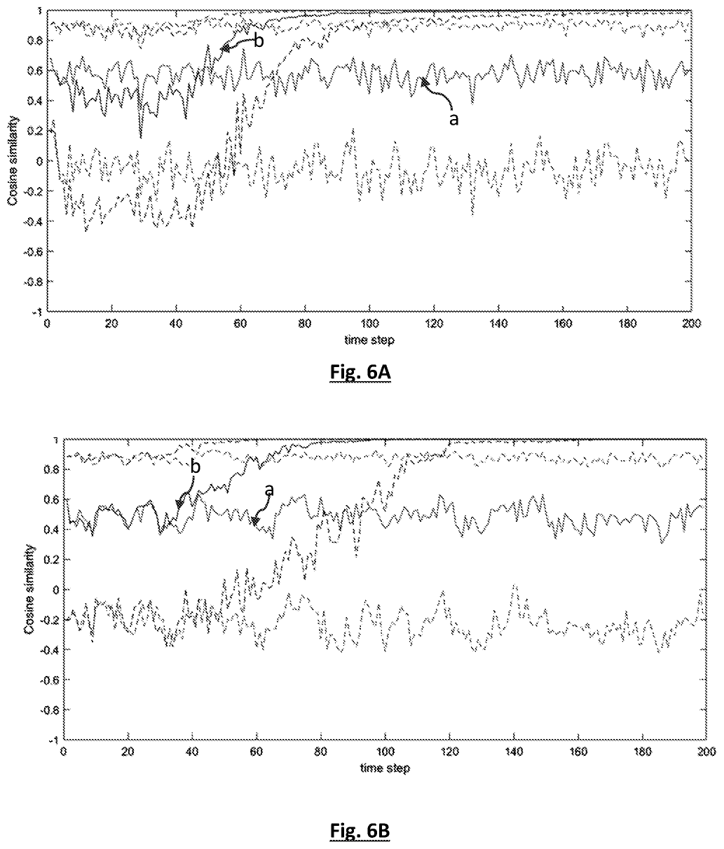

FIG. 5A shows the variation, as a function of time, in the distance between the effector and the target (curve a) and the distance between the estimated target and the actual target (curve b), considering the first noise model. The x-axis corresponds to time and the y-axis corresponds to the studied distances.

FIG. 5B shows the variation, as a function of time, in the distance between the effector and the target (curve a) and the distance between the estimated target and the actual target (curve b), considering the second noise model. The x-axis corresponds to time and the y-axis corresponds to the studied distances.

In FIGS. 5A and 5B, the dashed curves represent the limits of the confidence intervals.

In both figures, it may be seen that the target position stabilizes when the effector is sufficiently close to the target. s≥60 in FIG. 5A and s≥40 in FIG. 5B

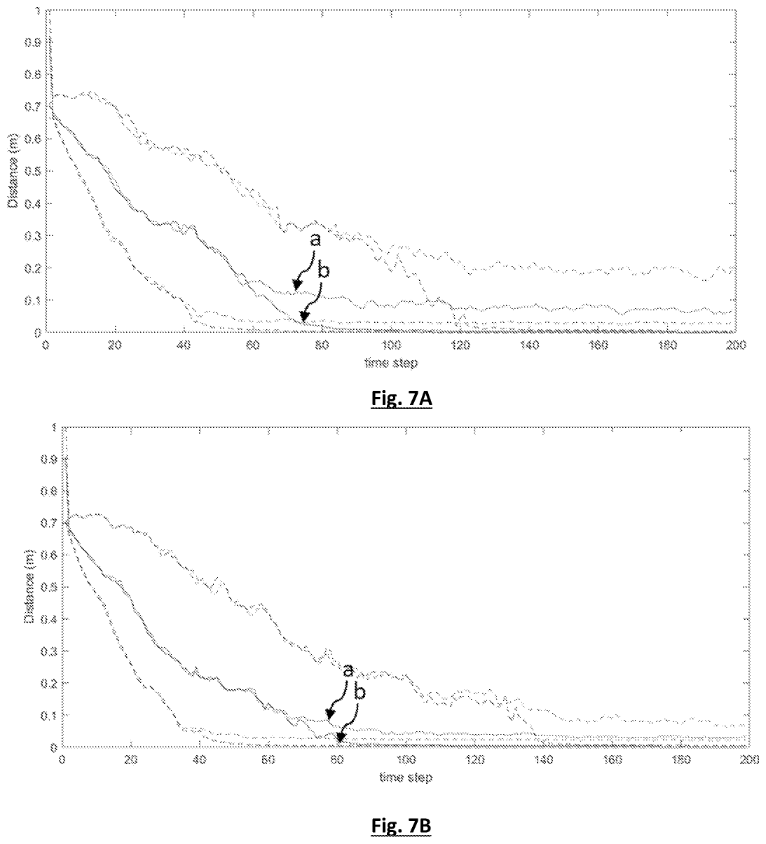

FIG. 6A shows the variation, as a function of time, in the angular accuracy

computed taking into account the predictive model (curve a) and the estimation of the target (curve b), considering the first noise model. The x-axis corresponds to time and the y-axis corresponds to angular precision, expressed in the form of a cosine similarity.

Curve a corresponds to CosSim(x_{d}^{s} , x_{d}^{p} ) and curve b corresponds to CosSim(x_{d}^{s}, x_{p,MPT}^{s}). This curve makes it possible to compare the prediction performance:
of the predictive model alone (curve a), this amounting to implementing (7) with k=0;

of the estimation of the target position, this amounting to implementing (7) with k=1.

FIG. 6B shows the variation, as a function of time, in the angular accuracy computed taking into account the predictive model (curve a) and the estimation of the target (curve b), considering the second noise model. The x-axis corresponds to time and the y-axis corresponds to angular precision, expressed in the form of a cosine similarity. Just as in FIG. 6A, curve a corresponds to CosSim(x_{d}^{s}, xₚ^{s}) and curve b corresponds to CosSim(x_{d}^{s}, x_{p,MPT}^{s}).

It may be seen that the angular accuracy obtained with estimation of the target by the method proposed here (curves b) is better than the angular accuracy resulting from the predictive model, provided that the effector is sufficiently close to the target: s≥60 in FIG. 6A (cf. FIG. 5A) and s≥40 in FIG. 6B (cf. FIG. 5B).

FIGS. 7A to 7D show the variation, as a function of time, in the distance between the effector and the actual target (curve a) and the distance between the estimated target and the actual target (curve b), considering the second noise model. The x-axis corresponds to time and the y-axis corresponds to the studied distances. The value of k was varied in the various figures: FIG. 7A: k=0.9; FIG. 7B: k=0.7; FIG. 7C: k=0.5; FIG. 7D: k=0.

Decreasing k makes it possible to better stabilize the effector position in proximity to the target, at the cost of a certain delay in terms of convergence.

The curves of FIGS. 7A to 7D may be compared with FIG. 5B, the latter corresponding to a movement of the effector without the estimated target position being taken into account (k=1).

### Three-dimensional Embodiment

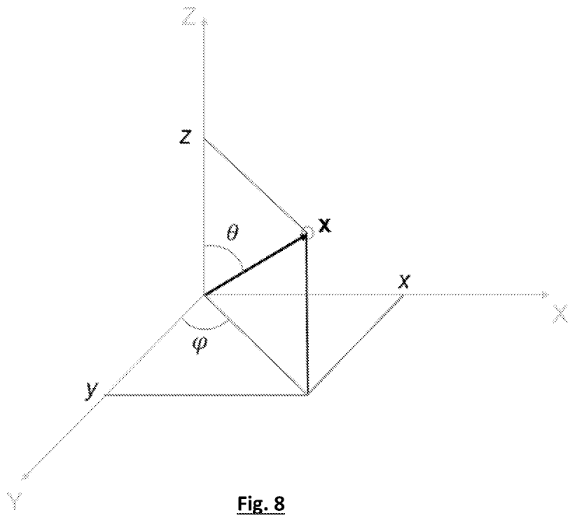

The invention has been described considering a two-dimensional effector movement. The same principles may be applied in three dimensions. FIG. 8 shows the angles to be taken into account, in a three-dimensional coordinate system, in which each position x is defined by three coordinates (x, y z)

$$φ = arctan ( \frac{y}{x} ) \qquad ( 15 )$$

$$θ = arctan ( \frac{\sqrt{{x}^{2} + {y}^{2}}}{z} ) \qquad ( 16 )$$

The angular errors between two vectors x₁, of coordinates (x₁, y₁, z₁) and x₂ of coordinates (x₁, y₁, z₁) are such that:

$${α}_{φ} ( {x}_{1} , {x}_{2} ) = arctan ( \frac{{y}_{1}}{{x}_{1}} ) - arctan ( \frac{{y}_{2}}{{x}_{2}} ) and \qquad ( 17 )$$

$${α}_{θ} ( {x}_{1} , {x}_{2} ) = arc tan ( \frac{\sqrt{{x}_{1}^{2} + {y}_{1}^{2}}}{{z}_{1}} ) - arctan ( \frac{\sqrt{{x}_{2}^{2} + {y}_{2}^{2}}}{{z}_{2}} ) \qquad ( 18 )$$

Assuming that the user is seeking to decrease the angular errors in each time interval, and that the angular errors follow independent Gaussian distributions of same power, the target probability density, defined in 2D in expression (3), becomes:

$$P ( x = {x}_{tg}^{tr} \vert {x}_{c}^{s} , {x}_{p}^{s} ) = \frac{1}{2 {πσ}^{2}} {e}^{- \frac{{{α}_{φ} ( {x}_{c}^{s} , - x , {x}_{p}^{s} )}^{2} - {{α}_{θ} ( {x}_{c}^{s} - x , {x}_{p}^{s} )}^{2}}{2 {σ}^{2}}} \qquad ( 19 )$$

Steps 100 to 180, described above, may be implemented in 3 dimensions based on the target probability density defined in (19).

1. A method for controlling the movement, through a space, of an effector via a brain-computer interface, the movement tending to bring the effector closer to a target position, the method comprising:
a) acquiring electrophysiological signals produced in the cortex of an individual, at a measurement time, the effector occupying a position, in the space, at said measurement time;
b) processing the electrophysiological signals, to form input data;
c) processing the input data, by means of a predictive model implemented by the brain-computer interface, to define an effector movement at the measurement time;
d) determining a corrected effector movement, based on the movement defined by the predictive model in step c);
e) controlling the movement of the effector through the space, by means of the brain-computer interface, based on the corrected movement resulting from step d);
f) incrementing the measurement time and reiterating steps a) to e) until a criterion for ending the iterations is met;
the method comprising, in each step d) following at least a first iteration of steps a) to e):
di) estimating the target position, at the measurement time, based on at least:
an effector position at the measurement time and at least one time preceding the measurement time;
a movement defined by the predictive model at the measurement time and at least at the time preceding the measurement time;
dii) based on the target position estimated in sub-step di), determining a movement towards the estimated target;
diii) taking into account the movement towards the estimated target, resulting from sub-step dii), to establish the corrected effector movement at the measurement time.

2. The method of claim 1, wherein sub-step di) comprises:
computing a target probability density, corresponding to a probability density reflecting the probability that each point in the space corresponds to the target position;
determining the point in the space maximizing the target probability density, or a quantity comprising the target probability density, the point thus determined corresponding to the estimated target position.

3. The method of claim 1, wherein computing the target probability density takes into account:
a plurality of effector positions at times preceding the measurement time;
a plurality of movements defined by the predictive model at times preceding the measurement time.

4. The method of claim 3, wherein the target probability density is computed based on a combination:
of a first conditional probability density; dependent on:
effector positions at times preceding the measurement time;
movements towards the estimated target defined by the predictive model at times preceding the measurement time;
and of a second conditional probability density P(x=x_{tg}x_{c}^{s}, xₚ^{s}) dependent on:
the effector position at the measurement time;
the movement towards the estimated target defined by the predictive model at the measurement time.

5. The method of claim 4, wherein the first conditional probability density is weighted by a forgetting factor (μ).

6. The method of claim 2, wherein sub-step di) comprises determining the point maximizing a quantity comprising the target probability density, the quantity corresponding to the target probability density decreased:
by a first distance (∥ [symbol] −x∥), weighted by a first regularization factor (λ₁), between each point and the target position estimated at a preceding measurement time;
and/or by a second distance (∥x_{c}^{s}−x∥), weighted by a second regularization factor (λ₂), between each point and the effector position at the measurement time.

7. The method of claim 1, wherein, in sub-step dii), the movement towards the estimated target is established so that the interface moves the effector from the position at the measurement time to the target position estimated at the measurement time.

8. The method of claim 1, wherein, in sub-step diii), the corrected movement is a combination of the movement defined by the predictive model in step b) and of the movement towards the estimated target resulting from sub-step dii).

9. The method of claim 8, wherein the combination employs:
a first weighting factor applied to the movement defined by the predictive model;
a second weighting factor applied to the movement towards the estimated target.

10. Brain-computer interface, comprising:
sensors, configured to acquire electrophysiological signals representative of a cortical activity;
an effector, configured to be actuated by a control signal generated by the brain-computer interface;
a processor programmed to determine an effector movement depending on input data resulting from the detected electrophysiological signals;
the processor being configured to implement steps c) to f) of a method according to claim 1, at various measurement times.
